(19) United States
(12) Patent Application Publication
(10) Pub. No.: US 2007/0172842 A1
(43) Pub. Date: Jul. 26, 2007

(54) Methods for isolating and identifying novel target-specific structuremers for use in the biological sciences

(76) Inventor: Andreas Braun, San Diego, CA (US)

Correspondence Address: BIOTECHNOLOGY LAW GROUP;C/O PORTFOLIOIP, PO BOX 52050, MINNEAPOLIS, MN, 55402, US

(21) Appl. No.: 11/340,379

(22) Filed: Jan. 25, 2006

(51) Int. Cl. C12Q 1/68

(52) U.S. Cl. 435/6

(57) ABSTRACT

Described are methods for isolating and identifying structuremers that specifically associate with non-nucleic acid target molecules. Each such structuremer species preferably minimally comprises a nucleic acid mimic capable of hybridizing to a substantially complementary single-stranded nucleic acid molecule under stringent hybridization conditions. Structuremers may also include tags to facilitate separation from other reaction components. Structuremer molecules identified by these methods will find application in many fields, including therapeutics and diagnostics.

### RELATED APPLICATIONS

This non-provisional patent application claims the benefit of, and priority to, each of the following patent applications: U.S. provisional patent application Ser. No. 60/490,206, filed 25 Jul. 2003; and PCT patent application Ser. No. PCT/US2004/023948, filed 23 Jul. 2004, each of which has the same title as this patent application, and each of which is hereby incorporated by reference in its entirety for all purposes.

### TECHNICAL FIELD

This invention concerns methods of identifying molecules that specifically interact with targets of interest, as well as products that comprise molecules identified using such methods.

### BACKGROUND OF THE INVENTION

1. Introduction

The following description includes information that may be useful in understanding the present invention. It is not an admission that any such information is prior art, or relevant, to the presently claimed inventions, or that any publication specifically or implicitly referenced is prior art, and nothing herein is to be construed as an admission that the invention is not entitled to antedate such disclosure by virtue of prior invention.

2. Background

One consequence of the human genome project is an accelerated effort to understand the genetic components involved in complex human disorders. Researchers are applying a variety of different strategies and technologies to tackle this important scientific and commercial task. The main strategies target analytes at distinct levels of cellular complexity, viz., the genome, transcriptome, proteome, and metabolome molecular levels. Each of these levels presents distinct technological challenges and requires different analytical methods, with the consequence that, at present, a single platform technology has not successfully fulfilled all the requirements.

A major drawback of testing for disease-associated genetic variation is that it suffers from low specificity caused by, among other factors, variable penetration of assayed genetic alterations among individuals of the same population. In contrast, proteins and metabolites show a much higher disease specificity since either or both of these classes of molecules directly leads to, is involved in, or results from a particular clinical phenotype. Also, many diseases are polygenic such that detection of a single gene, allele, or genetic variant is only weakly correlated with the existence of a particular disease, whereas the presence of a known disease-associated protein or metabolite in a tissue sample from a patient more strongly correlates with the clinical manifestation of the particular disease.

The proteome and metabolome levels of cellular complexity (the study of which are referred to as “proteomics” and “metabolomics”, respectively) each represent distinct areas where the molecular expressions of disease pathology can be analyzed. These levels can be considered as specific sites where genomic information is executed (by proteins) and represented in the context of specific products (ie., metabolites) found only in various disease states. A major challenge remains the rapid, flexible, and cost-effective generation of highly specific reagents to specifically detect the multitude of disease-specific proteins, metabolites, and other biomolecules. Access to such reagents would be important in areas such as life science research, diagnostics, and disease management and treatment.

As discussed above, proteins and metabolites are perhaps more appropriate diagnostic targets for common diseases than are DNA-based genetic tests, which are perhaps better suited for predicting disease susceptibility, drug efficacy and/or safety, detecting infection by a pathogen, etc. Reflecting this preference, to date almost all diagnostic tests target protein presence, protein amount, protein function, or pathway-specific metabolites. Proteomic and metabolomic strategies may also be valuable to differentiating between genetic and environmental influences that might contribute to disease etiology.

Despite the aforementioned limitations to genetic testing, sample preparation for genomic diagnostic approaches is relatively easy for several reasons: (i) the simplicity of DNA/RNA building blocks (only five widely used nucleotides, namely those containing the bases adenine, cytosine, guanine, thymine, and uracil), (ii) straightforward methods for the synthesis of specific reagents (e.g., oligonucleotides) and isolation of nucleic acids, and (iii) the existence of robust, widely-used amplification procedures (e.g., PCR). With regard to metabolites, a few extraction methods allow the analysis of a broad range of metabolites present in biological samples from a variety of different sources. The situation is much more complex with regard to proteins. While numerous protein purification techniques exist, all are cumbersome, time consuming, and reagent-specific, and experience demonstrates that the requirement for specific protocols regarding the particular protein being purified. For these reasons, such procedures seldom satisfy the requirements for high-throughput research or diagnostic applications. Also, a significant need remains for technology for the rapid generation of highly specific reagents to facilitate purification (e.g., protein-specific reagents for affinity chromatography) and further analysis/characterization of proteins and their function. Today, monoclonal antibodies, phage display, and high affinity nucleic acid ligands all play major roles in generating specific reagents for proteins and other target molecules. Each of these classes of reagents has various advantages and disadvantages, as discussed below.

Monoclonal antibody technology had its genesis in the mid-1970s (Kohler and Milstein, Nature 256: 495-497; 1975). Although epitope-specific monoclonal antibodies can be genetically engineered (e.g., “humanized”) and mice or even plants can be made to produce human antibodies at large scale, their production still requires several weeks or months and reliance upon complex biological systems. Such factors make the use of monoclonal antibodies, particularly in research environments, rather inflexible and too costly. It must also be appreciated that monoclonal antibodies are large biological molecules that are not resistant to degradation in many systems. This is disadvantageous for research, in vitro diagnostics, as well as in vivo therapeutic applications. Also, due to their size and the manner in which they are processed in biological systems, monoclonal antibodies themselves cannot be used to treat organisms on the intracellular level, although they have been shown in several instances to be useful as targeting agents for cell- or tissue-specific delivery of a potential therapeutic agent (e.g., a small molecule drug, a therapeutic protein, a gene delivery vehicle, etc).

“Phage display” technologies began to appear in the mid-1980s (Smith, Science 228: 1315-1317; 1985), wherein a virion is engineered to present a foreign amino acid sequence in immunologically accessible form as a component in a coat protein of a viral particle. This approach has been used to individually display many discrete regions of different peptides and proteins, including human antibodies and enzymes, on the surface of a small bacterial virus. Phage display affords a method for producing and searching through large collections, or libraries, of peptides and proteins to rapidly identify those that might bind with high affinity and high specificity to targets of interest. However, peptides and proteins identified by such methods are also biologically degradable, and frequently it has been shown that peptides so identified have only a very low affinity to the corresponding target molecules. Subsequent evolution of such peptides by chemical modifications or the transformation to peptoids often further weakens their already initially poor target specificity and affinity.

In 1990, the laboratories of G. F. Joyce (Robertson and Joyce, Nature 344: 467-468; 1990), J. W. Szostak (Ellington and Szostak, Nature 346: 818-822; 1990), and L. Gold (Tuerk and Gold, Science 249: 505-510; 1990) independently reported development of a technique that allows the simultaneous screening of more than 10¹⁵ individual nucleic acid molecules for different functionalities. This method is now commonly known as “in vitro selection”, “in vitro evolution”, or “SELEX” (systematic evolution of ligands by exponential enrichment). This technique has since become a useful tool in molecular biology. Using an in vitro selection-technique, large random pools of nucleic acids can be screened for a particular functionality, such as the binding to small organic molecules or large proteins, as well as for the presence of a desired catalytic activity, e.g., ribozyme activity. Nucleic acids having the desired function can be selected from the mainly non-functional pool of RNA or DNA by column chromatography or other selection techniques suitable for the enrichment of the desired property. The functional molecules, i.e., those nucleic acids having the desired activity, have sometimes been referred to as “aptamers” (a linguistic chimera derived from a combination of the Latin “aptus”, which means “to fix”, and the Greek suffix “mer”). The conventional in vitro selection method is conceptually straightforward: a standard, automated DNA-oligonucleotide synthesizer is used to generate a starting pool of different nucleic species. Each species is distinguished by its unique nucleotide sequence, which is derived from the machine synthesis of oligonucleotides with completely or partially random nucleotide sequences. Defined primer binding sites flank the random regions to facilitate later amplification, provided that the desired activity is detected upon assaying. In this way, up to 10¹⁵ different DNA molecules may be synthesized at once in a common pool. As will be appreciated, such a pool is an incredibly complex mixture, particularly when one considers that the number of distinct antibody species a mouse can possibly generate has been estimated to be between about 10⁹ to 10¹¹ species. The immense complexity of such a synthetic pool of nucleic acids may justify the assumption that it may contain a few molecules with the correct secondary and/or tertiary structures to confer the desired activity, e.g., catalysis of a specific RNA molecule (in the case of a ribozyme) under the conditions assayed. If present, active species may be selected, for example, by affinity chromatography or filter binding. Because a pool of such high complexity (in terms of the number of distinct nucleic acid species present) can be expected to contain only a very small fraction of functional molecules, several purification steps are usually required. To accomplish this, the very rare active aptamer molecules, if any, are typically themselves amplified by a technique such as the polymerase chain reaction (“PCR”; see, e.g., U.S. Pat. Nos. 4,683,195; 4,683,202; 4,800,159; 4,965,188; 5,176,995; 5,187,083; 5,219,727; 5,234,824; 5,333,675; 5,468,613; 5,604,099; and 5,656,493), in a transcription-based amplification process (see, e.g., U.S. Pat. Nos. 5,399,491; 5,399,491; 5,480,784; 5,554,516; 5,766,849; 5,824,518; 5,846,701; 5,871,975; 5,888,779; 6,087,133; 6,214,587; 6,294,338), by strand displacement amplification, or by any other suitable amplification method. In this way, iterative cycles of selection can be carried out. Successive selection and amplification cycles result in an exponential increase in the abundance of functional sequences, until they dominate the population, at which time the nucleotide sequences of the functional aptamers can be obtained via conventional nucleic acid sequencing techniques. After sequencing, a functional aptamer can be synthesized on any desired scale. The method has reportedly been applied in a number of different applications, and there have been several reported discoveries of aptamers that not only bind tightly to proteins, but also inhibit their biological activity. That said, despite the emergence of aptamer technology more than ten years ago, to date there appear to be no reports of commercial products developed using aptamer technology.

As the foregoing makes clear, there is a clear need for technologies that enable the discovery of compounds that can specifically bind to target molecules of interest. Recent advances in proteomics and metabolomics make this need even more pressing, as the number of molecules known to be biologically relevant is increasing tremendously.

3. Definitions

Before describing the instant invention, several terms used in connection with its description will be defined. In addition to these terms, others are defined elsewhere in the specification, as necessary. Unless specifically defined otherwise in this specification, all technical and scientific terms used herein have the same meanings as commonly understood by one of ordinary skill in the art to which this invention belongs.

The term “structuremer” as used herein refers to synthetic molecules that specifically bind to target molecules that are other than naturally occurring nucleic acids. Structuremers also hybridize to substantially complementary polynucleotides that may show little if any target specificity, and the nucleotide sequence of which reveals the sequence of base-binding moieties in the structuremer. In the presence of a specific target molecule, structuremers specific for the target molecule distinguish themselves from non-specific structuremers based on their ability to specifically bind to the target molecule. Structuremers are also non-amplifiable, i.e., they cannot be extended by nucleotide-dependent polymerase, and they do not function as templates for the initiation of transcription.

A “nucleic acid mimic” refers to linear or cyclic single-stranded molecule that is incapable of replication or amplification by an in vitro or in vivo biological system and comprises an array of hydrogen bond donors and acceptors capable of preferentially hybridizing to a single-stranded polynucleotide sufficiently complementary thereto. Preferably, the hydrogen bond donors and acceptors are provided by a plurality base moieties arrayed from a polymerization scaffold in a manner that provides a spatial orientation sufficiently duplicative of a nucleic acid molecule capable of forming a nucleic acid duplex with a complementary single-stranded polynucleotide. A “linear” nucleic acid mimic refers to a molecule wherein the base moiety at each of the proximal and distal ends is linked to only one other base moiety (a succeeding base moiety in the case of the base moiety at the proximal end, and a preceding base moiety in the case of the base moiety at the distal end), and no there is no cross-linking or other direct or indirect (i.e., through a linker) attachment of one base moiety to another in the particular molecule. A “cyclic” nucleic acid mimic refers to one wherein one base moiety is cross-linked or otherwise directly or indirectly attached to another base moiety of the same nucleic acid mimic.

In the context of this invention, a “base” refers to any chemical moiety that can be included within a nucleic acid molecule or nucleic acid mimic without disrupting the structure of the molecule sufficiently to prevent hybridization with a complementary nucleic acid molecule. Preferably, although not necessarily, a base provides at least one hydrogen bond donor and/or hydrogen bond acceptor for purposes of hybridizing to a base in a complementary molecule, be it in a nucleic acid or a nucleic acid mimic of the invention. In the double helical structure of naturally occurring nucleic acids, the base adenine hydrogen bonds with the base thymine or uracil, the base guanine hydrogen bonds with the base cytosine, and the base inosine hydrogen bonds with adenine, cytosine, or uracil. At any point along the chain of a double-stranded nucleic acid, therefore, one may find the classical “Watson-Crick” (“canonical”) base pairs A:T or A:U, T:A or U:A, and G:C or C:G. One may also find A:G, G:U, and other “wobble” or mismatched base pairs in addition to the canonical base pairs. Preferred bases include A, G, C, T, U, and I. When bases other than A, T, U, C, or G are incorporated into a nucleic acid molecule, other base pairing may occur. Such bases may be specific for a particular base, or they may confer specificity for two or more other bases, including non-naturally occurring bases. The decision of whether to include such bases, and, if so, which base(s) and at what position(s), in structuremers of the invention is left to the discretion of the skilled artisan.

Herein, the term “base moiety” refers to nucleosides and nucleotides used to make nucleic acids (e.g., oligonucleotides), as well as non-nucleoside and non-nucleotide subunits or moieties that can be used to make the nucleic acid mimic components of structuremers. In general, the base moiety comprises a base and a polymerization scaffold. Typically, the base is covalently linked to the polymerization scaffold, as is the case with nucleosides and nucleotides.

A “polymerization scaffold” is a chemical moiety having at least three sites that can be, or are, derivatized. In the context of the invention, one of the sites is derivatized by covalent attachment of a base, directly or through a linker. The other two sites (each a coupling group) are used for linkage to the polymerization scaffold of a preceding or succeeding base moiety (i.e., the immediately preceding base moiety or immediately succeeding base moiety, respectively, when viewing the nucleic acid mimic from proximal to distal end).

An “amino acid” is a molecule having the structure wherein a central carbon atom (the “alpha (α)-carbon atom”) is linked to a hydrogen atom, a carboxylic acid group (the carbon atom of which is referred to as a “carboxyl carbon atom”, and the oxygen of which that is not lost in a subsequent polymerization reaction is referred to as the “carbonyl oxygen atom”), an amino group (the nitrogen atom of which is referred to as an “amino nitrogen atom”), and a side chain group, R. In the process of being incorporated into a protein, an amino acid loses one or more atoms of its amino and carboxylic groups in a dehydration reaction that links one amino acid to another. As a result, when incorporated into a protein, an amino acid is often referred to as an “amino acid residue.” By analogy, a base moiety, after incorporation into a structuremer, may be referred to as a “base moiety residue”. An amino acid may be one that occurs in nature in proteins, or it may be non-naturally occurring (:ie., is produced by synthetic methods such as solid state and other automated synthesis methods). Examples of non-naturally occurring amino acids include alpha-amino isobutyric acid, 4-amino butyric acid, L-amino butyric acid, 6-amino hexanoic acid, 2-amino isobutyric acid, 3-amino propionic acid, ornithine, norlensine, norvaline, hydroxproline, sarcosine, citralline, cysteic acid, t-butylglyine, t-butylalanine, phenylylycine, cyclohexylalanine, beta-alanine, fluoro-amino acids, designer amino acids (e.g., beta-methyl amino acids, ac-methyl amino acids, N-alpha-methyl amino acids), and amino acid analogs in general.

An “amino acid core” refers to an amino acid exclusive of its R-group, with or without reference to hydrogen atoms. Thus, an amino acid core of a base moiety using the core as its polymerization scaffold refers to the α-carbon atom, the carboxyl carbon atom, the carbonyl oxygen atom, and the amino nitrogen atom. In the context of such a base moiety in, or to be included in, a nucleic acid mimic, the base serves as the R-group, which may be attached directly to the amino acid core via a covalent linkage, or indirectly to the core by way of a linker. Base moieties that comprise an amino acid core and a base linked thereto comprise but one representative class of base moieties useful in the practice of the invention.

A “peptide nucleic acid” or “PNA” refers to a preferred class of structuremers. PNAs are nucleic acid analogues DNA in which the backbone is a pseudopeptide rather than a series of polymerized sugar molecules linked by various chemistries. PNAs mimic the behavior of nucleic acids and binds complementary nucleic acid strands. The neutral backbone of PNA results in stronger binding and greater specificity for complementary nucleic acids, as compared to nucleic acids comprised of polymerized nucleosides. In analogy to DNA, sequence complementary PNAs are known to form duplex molecules.

A “polynucleotide” or “nucleic acid” may be either RNA or DNA unless specified otherwise. It is well established that two single strands of deoxyribonucleic acid (“DNA”) or ribonucleic acid (“RNA”) can associate or “hybridize” with one another to form a double-stranded structure having two strands held together by hydrogen bonds between complementary base pairs. The individual strands of nucleic acids are formed from nucleotides that comprise the bases such as adenine (A), cytosine (C), thymine (T), guanine (G), uracil (U), and inosine (I).

A “nucleotide” is the basic monomeric building block, or subunit, of certain nucleic acids. A nucleotide comprises at least one phosphate group, a 5-carbon sugar, and a nitrogenous base. In naturally occurring nucleic acids, the sugar groups contain five carbons, with the 5-carbon sugar found in RNA being ribose (being comprised of ribonucleotides) and 2′-deoxyribose in DNA (DNA being comprised of deoxyribonucleotides). In nature, the sugar of a 5′-nucleotide typically contains a hydroxyl group (—OH) at the 5-carbon position. However, as used herein, the term also includes analogs of naturally occurring nucleotides, such as analogs having a methoxy group at the 2′ position of the sugar (OMe), as well as other moieties such as pyranosyl RNA monomers. Bases include A, G, C, T, U, and I. The term includes nucleotides having one, two, or three phosphate groups (mono-, di-, and tri-phosphates, respectively). Naturally occurring nucleic acids are formed by the polymerization of individual nucleotide subunits through the formation of phosphodiester bonds between the sugar moieties of the nucleotides. Non-phosphorylated moieties comprising bases and sugars are termed “nucleosides.”

An “oligonucleotide” is a polynucleotide having two or more nucleotide subunits covalently joined together, although the term will also be understood to include nucleotide/non-nucleotide polymers to the extent the same are used for the purpose of detecting and/or amplifying a target-specific structuremer. Oligonucleotides generally have a length of from 5 to about 200 nucleotides, preferably from about 10 to bout 100 nucleotides, although oligonucleotides of greater length can be generated. Ordinarily, oligonucleotides are synthesized by organic chemical methods, and they are single-stranded unless specified otherwise. Oligonucleotides may be labeled with a detectable label. The term also includes analogs of naturally occurring nucleotides, particularly those having a methoxy group at the 2′ position of ribose (OMe). The nucleotide subunits may by joined by linkages such as phosphodiester linkages, modified linkages, or by non-nucleotide moieties that (i) do not prevent hybridization of the oligonucleotide to its complementary target sequence or (ii) contribute to hybridization by providing hydrogen bond donors and acceptors arrayed in three-dimensional space in a manner that promotes hydrogen bond formation with corresponding hydrogen bond acceptors and donors with the bases of a complementary molecule. Modified linkages include those in which a standard phosphodiester linkage is replaced with a different linkage, such as a phosphorothioate linkage, a methylphosphonate linkage, a formacetal linkage, a morpholino linkage, a sulfamate linkage, a carbamate linkage, or a neutral peptide linkage. Nitrogenous base analogs also may be components of oligonucleotides in accordance with the invention. Examples of oligonucleotides useful in the context of the invention include amplification primers and probe oligonucleotides.

An “amplification primer” is an oligonucleotide designed to hybridize to a primer-binding site (i.e., an engineered nucleotide sequence designed to facilitate primer binding and subsequent amplification) in a nucleic acid, such as an oligonucleotide. Depending on the amplification process being employed, the primer may be extended in the amplification reaction. Alternatively, it may serve simply to initiate transcription. Amplification primers may contain sequences in addition to that designed to bind to a primer-binding site. Examples of such additional sequences are promoters for an RNA polymerase. Useful promoters include the T7 and SP6 promoters.

A “probe” oligonucleotide is an oligonucleotide than comprises at least one, and preferably two, primer-binding regions and a “probe region” for hybridizing to a complementary nucleic acid mimic. Thus, a probe single-stranded nucleic acid molecule that contains a region that allows it to hybridize to a complementary nucleic acid mimic under stringent conditions, as well as one or more regions that facilitate later amplification. “Amplifying” means increasing the number of copies of a particular polynucleotide. Amplification can be accomplished by any suitable technique, including amplification in vivo (ie., by cloning) and in vitro.

A “biologically active” form of a biological molecule (e.g., a protein, carbohydrate, or lipid (including metabolites thereof)) refers to a form of the molecule having the structural, immunological, regulatory, or chemical function of a naturally occurring or engineered form of the molecule, as the case may be.

By “complementary” is meant that a nucleic acid (e.g., an oligonucleotide primer) can hybridize to a region of the nucleic acid template or nucleic acid mimic under the conditions of the contacting, combining, or reaction mixture forming step, as the case may be, thereby facilitating formation of structuremer/nucleic acid complexes, primer/template complexes, and the like. For example, a “complementary primer” refers to an oligonucleotide primer in which at least a portion of its nucleic acid sequence is complementary to a nucleic acid sequence present on a nucleic acid template, while an oligonucleotide or other nucleic acid complementary to a nucleic acid mimic refers to an oligonucleotide having a nucleotide sequence complementary to the bases of the nucleic acid mimic when the two molecules are aligned in a manner which allows hybridization between their respective regions of complementarity. Generally, nucleic acids useful in the context of the invention will have a region of complementarity (to other nucleic acids and/or nucleic acid mimics) to the target sequence of between about 8 and about 100 bases, preferably between about 12 and 50 bases, although any region of complementarity sufficient to accomplish the desired end may be employed.

The term “complementary” may also be used in the context of single bases, for example, adenine is complementary with thymine and uracil, and guanine is complementary with cytosine. “Complementarity” is a property conferred by the base sequence of a single stranded polynucleotide or nucleic acid mimic that enables formation of a duplex with a complementary nucleic acid (e.g., an oligonucleotide) through hydrogen bonding, typically between bases on the respective strands. A “mismatch” refers to any pairing in a hybrid of two bases that do not form at least one canonical Watson-Crick hydrogen bond. As will be appreciated, a mismatch can also result from an insertion or deletion in one strand of the hybrid that results in one or more unpaired bases. It will be appreciated that the degree of complementarity between strands of duplex may vary. In the cases where hybridization between strands of the duplex results from hydrogen bond formation between bases in the two strands, such variation may be stated in terms of a percentage of complementary bases between the strands in the regions of complementarity. Preferably, the region of complementarity is 100% complementary over its length, as compared with the region of complementarity of the other strand. That is, over the regions of complementarity between two single-stranded molecules, each base in one of the single strands can hydrogen bond with a base present on the other single strand, particularly under stringent hybridization conditions. However, those of ordinary skill in the art will recognize that single-stranded molecules having a region of complementary of less than 100% can also operate efficiently in the practice of the invention. Generally, such complementarity can be as few as 13 out of 18, preferably not less than about 15 out of 18, contiguous bases. In preferred embodiments, the percentage of complementarity is at least about 85%. In more preferred embodiments, this percentage is from about 90% to 100%; in other preferred embodiments, this percentage is from about 95% to 100%. A “hybrid” or a “duplex” is a double-stranded, hydrogen-bonded complex formed between two single-stranded nucleic acid molecules or a single-stranded nucleic acid molecule (preferably an oligonucleotide) and a single-stranded nucleic acid mimic, by Watson-Crick base pairings or non-canonical base pairings between complementary bases in two molecules. Each such base pairing is a “base pair”. Preferably, duplexes are from about 8 to about 200 base pairs in length. More preferably, duplexes range from about 10 to about 100 base pairs in length. It will be understood that in the context of duplex length, “base pairs” includes mismatched base pairs, if any, in the event the two strands of the duplex are less than 100% complementary over their regions of complementarity. A “stable” hybrid refers to one that can remain intact under stringent hybridization conditions. “Hybridization” is the process by which two complementary single-stranded molecules (be they polynucleotides or a polynucleotide and a nucleic acid mimic) form a hybrid or duplex.

The terms “contacting”, “combining” reagents to “form a reaction mixture”, and the like mean that the various reagents and reactants required for a particular reaction are brought together under conditions that allow the reaction to occur. For example, in the context of nucleic acid amplification, “contacting” or “combining” means that a nucleic acid template, a nucleic acid polymerase, the required nucleotides and other chemicals (e.g., salts, co-factors, etc.), and suitable oligonucleotide primers are brought together under primer extension conditions.

“Fragment” refers to any part of a molecule that retains a usable, functional characteristic of the molecule from which the molecule is derived. A “portion” of a target molecule, as used herein, refers to any part of a protein, lipid, carbohydrate, or metabolite used for any purpose, but especially for the screening of structuremers to identify one or more molecules that specifically associate with that portion of the target molecule.

A “label” is a detectable moiety that may be attached to the end(s), or, alternatively, at an internal position, of a nucleic acid mimic or oligonucleotide. Detectable moieties include radioisotopes, chemiluminescent molecules, fluorescent molecules, enzymes, haptens, quantum dots, or even unique oligonucleotide sequences.

In the context of hybridization, “stringency” describes the temperature and solvent composition existing during hybridization and the subsequent processing steps. Under stringent hybridization conditions only highly complementary duplexes will form; hybrids without a sufficient degree of complementarity will not form. Accordingly, the stringency of the assay conditions determines the amount of complementarity needed between the two strands forming the duplex. Stringency conditions are chosen to maximize the difference in stability between desired duplexes (e.g., between a nucleic acid mimic and a complementary oligonucleotide) and undesired duplexes.

The term “conditions that allow” refers to temperature, solvent, time factors, pH, ionic strength, and any other factor that affects molecular association or other reaction aspects. For example, “conditions that allow hybridization to occur” refer to those where duplexes can form. “Conditions” that allow a structuremer specific for the non-nucleic acid target molecule to associate with the target to form a structuremer/target complex refers to any set of conditions wherein such association can occur. Such conditions preferably are physiological conditions. As those in the art will appreciate, what constitutes “physiological conditions” depends on many factors, including the target molecule. Typically, physiological conditions for a given reaction will mimic the conditions of the biological system in which the non-nucleic acid target molecule is found in nature. For many systems, these conditions are known. For others, they can be derived using methods known in the art.

“Tm” refers to the “melting” temperature at which 50% or more of a single-stranded nucleic acid mimic or polynucleotide is converted from a hybridized to an unhybridized form.

A “linker” or like term refers broadly to any molecule that can be used to covalently link one molecule to another molecule. Preferably, linkers are aliphatic straight chains, although branched linkers may also be used. Such chains generally range from 2 to about 80 carbon atoms in length. The one or more of the carbon atoms in the chain may be joined to another carbon atom of the chain by single, double, or triple bond. A linker will typically have a functional group (also known as a conjugation partner or coupling group) at each end of the chain that is capable of reacting with another functional group in a different molecule. Together, these groups form a conjugation pair. Linkers intended to link to molecules preferably are bifunctional, in that they have a different functional group at each end of the linker to allow sequential assembly of the final molecule, first by linking the linker to one of the molecules using a chemistry suitable for the members of the particular conjugation pair, then by linking the other molecule to the linker:first molecule intermediate using a chemistry suitable for the other conjugation pair. Exemplary functional groups include amino, hydroxyl, sulfhydryl, and like functional groups.

An “agonist” is a compound that binds (covalently or non-covalently) to and modulates the activity of another molecule. An agonist can be a “negative agonist”, i.e., a compound that decreases activity, or a “positive agonist”, i.e., a compound that increases activity. An “antagonist” is a compound that competes with another compound in interacting with third molecule (e.g., a protein, lipid, or carbohydrate). Structuremers include agonists and antagonists.

The term “identifying a structuremer” means determining at least the sequence of bases of the molecule's nucleic acid mimic. Information regarding a structuremer's molecular weight, three-dimensional structure, etc., may also be determined, if desired, using any suitable technique, e.g., mass spectrometry, solution NMR, and powder and single crystal diffraction.

The term “modulate” refers to a change in activity (e.g., biochemical activity) or function (biological, chemical, or immunological) or other attribute (e.g., an ADME (i.e., administration, distribution, metabolism, and excretion) characteristic) of one molecule mediated by another molecule. The change may be an increase or enhancement (including initiation or activation) of, or a decrease or reduction (including abolition) in activity. Modulation may occur by covalent or non-covalent interaction. Non-covalent interactions include hydrophobic interactions, hydrophilic interactions, electrostatic interactions, van der Waals forces, and steric interactions.

A “modulator” refers to a compound that causes a change, e.g., an increase or decrease, in activity of a molecule, and is typically an agonist or antagonist. A modulator may act directly, for example, by interacting with the molecule whose activity the modulator alters. A modulator may also act indirectly, for example, by interfering with, i.e., antagonizing or blocking, the action of another molecule that causes an increase or decrease in activity of the molecule (e.g., a protein).

A “multimer” or “multimeric structuremer” refers to a plurality of structuremers that are attached, either covalently or through a non-covalent, high affinity association. Structuremer multimers include dimers, trimers, and larger multimers of the same or different structuremers. Structuremers may be “homo-multimers” (i.e., multimers of the same structuremers) and “hetero-multimers” (molecules that comprise at least one of each of two different structuremer species).

“Primer extension conditions” refer to conditions wherein template-dependent amplification initiated at an oligonucleotide primer can occur. Such conditions generally include provision of an appropriate buffer and, optionally, other chemicals that may stabilize the polymerase, or enhance primer extension reactions.

“Protein” refers to a naturally occurring or synthetic polymer of amino acids linked by peptide bonds, and includes peptides, fragments, and polypeptides. “Protein” also includes those proteins comprised of multiple subunits, whether or not one or more of the subunits are covalently linked to one or more other subunits.

The term “reaction mixture” means a solution containing the ingredients necessary for a desired reaction to occur.

“Sample” is used herein in its broadest sense, and includes a bodily fluid (e.g., blood, plasma, urine, cerebrospinal fluid, semen, and mucous), a soluble fraction of a cell preparation, media in which cells are cultured, organelles or membranes isolated or extracted from cells, cells, tissues, skin, hair, and the like.

“Secondary structure” refers to local conformation of covalently linked atoms of a molecule, for example, of a protein or polynucleotide. In the context of proteins, secondary structure makes reference to the peptide bonds and alpha-carbon linkages that string the amino acid residues of the protein together. Representative examples of secondary structures include alpha helices, parallel and anti-parallel beta structures, and structural motifs such as helix-turn-helix, beta-alpha-beta, the leucine zipper, the zinc finger, the beta-barrel, and the immunoglobulin fold. “Tertiary structure”, by contrast, concerns the three-dimensional structure of a protein, including the spatial relationships of amino acid side chains and atoms, and the geometric relationships of different regions of the protein. Thus, regardless of class, secondary structure shall be understood to refer to local conformations of covalently linked atoms, whereas tertiary structure refers to the spatial and/or geometric relationships of constituent elements (e.g., atoms, pseudoatoms, side groups, chemically reactive groups, etc.) of molecules.

A “species” refers to a molecule having a distinct chemical formula. A variety of molecular species are referred to in the context of the invention. For example, two oligonucleotides are said to represent different oligonucleotide species when they differ in nucleotide sequence, and structuremers that, for example, differ in base sequence or in the composition of their respective polymerization scaffolds represent different species.

“Specifically associate”, “specific association,” and the like refer to a specific, non-random interaction between two molecules, which interaction depends on the presence of structural, hydrophobic/hydrophilic, and/or electrostatic features that allow appropriate chemical or molecular interactions between the molecules.

“Specificity”, in the context of structuremers, refers to the ability of the molecule to associate with its target molecule with a high level of affinity. Affinity can be represented by any suitable measure, including association and dissociation constants. On the other hand, “selectivity” refers to the ability of a structuremer specific for one target molecule to distinguish that target from other target molecules. Depending upon application, for example, a therapeutic application versus a diagnostic or research application, the required level of specificity and selectivity may differ. Regardless, however, it is preferred that a structuremer that binds to a target molecule exhibit both specificity and selectivity, preferably at a high degree.

“Sufficiently complementary” or “substantially complementary” means duplexes having a sufficient amount of contiguous complementary bases to form, under stringent hybridization conditions, a hybrid that is stable for detection or isolation. “Preferentially hybridize” means that under stringent hybridization conditions an oligonucleotide can hybridize to a nucleic acid mimic that comprises a sufficient number of complementary bases, for example, arrayed in a structure that allows formation of a stable duplex between the probe oligonucleotide and the mimic without forming stable duplexes with non-complementary nucleic acid mimics.

A “tag” is a molecule that may attached to a nucleic acid mimic, oligonucleotide, or non-nucleic acid target molecule for the purpose of facilitating isolation and/or purification. Such molecules include one member of a high affinity binding pair (e.g., biotin or avidin), a ligand for a receptor, and antibodies (or fragments thereof).

A “target molecule” is any naturally occurring or synthetic molecule, other than a naturally occurring nucleic acid, present in a biological system for which it is desired to identify a molecule that specifically associates with or binds to it. Representative examples of target molecules are proteins, peptides, lipids, and carbohydrates, and metabolites of the foregoing, as well as vitamins, bacteria, viruses, cell organelles, and entire cells. Particularly preferred are “non-nucleic acid target molecules,” which are targets other than DNA or RNA.

A “target sequence” refers to the particular base sequence (or its complement) of a nucleic acid mimic or polynucleotide that is to be amplified. In the context of a nucleic acid mimic, deduction of the target sequence allows identification of a molecule that specifically binds to or associates with a non-nucleic acid target molecule. In the context of nucleic acid molecules, the target sequence refers to a sequence to be targeted by another polynucleotide. For example, a target sequence may be the sequence of nucleotides that are complementary to an amplification primer, which are termed “primer binding sites”. The target sequence includes the complexing sequences to which oligonucleotide primers useful in the amplification reaction can hybridize prior to extension by a DNA polymerase.

A “targeting element” refers to a molecule attached to or included within a structuremer that provides additional targeting capability in addition to that provided by the nucleic acid mimic(s). Preferred targeting elements are peptides that may enhance the selectivity and/or sensitivity of binding of the structuremer to its target molecule.

“Template” refers to a single-stranded nucleic acid molecule, such as DNA, RNA, or another linear molecule comprised of subunits that present hydrogen bond donors and acceptors complementary to the bases of a complementary nucleic acid strand which can serve as a substrate for the synthesis of a complementary nucleic acid molecule.

### SUMMARY OF THE INVENTION

The object of the invention is to provide rapid, efficient methods for the isolation and identification of molecules that specifically bind to desired target molecules, particularly non-nucleic acid target molecules.

Thus, in one aspect, the invention concerns methods for isolating one or more structuremers that specifically associate with a non-nucleic acid target molecule. Such methods involve combining a structuremer with a non-nucleic acid target molecule under conditions that allow structuremers specific for the target molecule to associate to form a structuremer/target complex. Typically, a pool containing a plurality of different structuremer species is combined in a reaction mixture containing the target molecule. If a structuremer species specific for the target molecule is present in the pool, structuremer/target complexes are formed. Such complexes can then be isolated from the other reaction components. Given the complexity of biological systems, it is generally preferred that screening assays be conducted in a high throughput format, so that a large number of different structuremer species can be screened for binding activity with the target molecule.

In preferred embodiments, the structuremer species used in such assays each comprises a nucleic acid mimic capable of hybridizing to a substantially complementary single-stranded nucleic acid molecule under stringent hybridization conditions. This allows the sequence of moieties, or residues, in the structuremer to be deduced by determining the nucleotide sequence of the complementary single-stranded nucleic acid molecule that hybridizes to the structuremer under stringent hybridization conditions. In preferred embodiments where the screening assays employ a plurality of different structuremer species, the different structuremer species are preferably distinguished by their nucleic acid mimics, with each structuremer species having a different nucleic acid mimic. As a result, the nucleic acid mimic can be distinguished according to the single-stranded nucleic acid molecule to which it hybridizes under stringent hybridization conditions.

In addition to a nucleic acid mimic, structuremers also preferably further contain one or more tags. A tag can be any molecule that facilitates detection, and preferably isolation, of the structuremer, alone or complexed with other molecules (e.g., a target molecule to which the structuremer specifically binds). Tags may be attached at any suitable position in a structuremer, although attachment to an end of the structuremer is preferred. A structuremer may also further comprise a targeting element. Targeting elements are preferably comprised of amino acids, which are preferably linked via peptide bonds. The amino acids may be naturally D- or L- amino acids, derivatives thereof, or non-naturally occurring amino acids. Targeting elements are typically linked to the end of a nucleic acid mimic, directly or through a linker. When two ore more nucleic acids are used in a structuremer, a targeting element, if included, may be positioned between the nucleic acid mimics or, alternatively, at one or the other end of the structuremer. In some embodiments, a structuremer comprises two or more targeting elements, which may be of the same or different species.

While some structuremers contain only one nucleic acid mimic, in other embodiments, the structuremer may contain two or more nucleic acid mimics. In such embodiments, the nucleic acid mimics may be of the same or different species. The plurality of mimics may or may not be separated by an intervening moiety.

In preferred embodiments, the nucleic acid mimics incorporated into structuremers comprise polymers of independently selected base moieties each capable of specific hybridization to a different base in a single-stranded nucleic acid molecule. Such polymers contain any number of base moieties, although polymers comprised of between about 7 to about 100 independently selected base moieties are preferred. In general, base moieties comprise a base linked to a polymerization scaffold. Preferred bases include adenine, guanine, cytosine, thymine, uracil, inosine, xanthine, and hypoxanthine, or a heterocyclic derivative, analogue, or tautomer thereof, including 8-azapurine, purines substituted at the 8 position with methyl or bromine, 9-oxo-N⁶-methyladenine, 2-aminoadenine, 7-deazaxanthine, 7-deazaguanine, 7-deazaadenine, N⁴, N⁴-ethanocytosine, 2,6-diaminopurine, N⁶, N⁶-ethano-2,6-diaminopurine, 5-methylcytosine, 5-(C³—C⁶)-alkynylcytosine, 5-fluorouracil, 5-bromouracil, thiouracil, pseudoisocytosine, 2-hydroxy-5-methyl-4-triazolopyridine, isocytosine, isoguanine, 7,8-dimethylalloxazine, and such other non-naturally occurring bases now known or later developed. Generally, for base moieties that are intended for inclusion in a nucleic acid mimic other than at one of the two ends, the polymerization scaffold comprises at least three locations for attachment of other moieties. Two of these attachment points are conjugation partners used for linking one base moiety to the next during synthesis of the polymer. The other location is for attachment of a base (or other moiety). Preferred polymerization scaffolds are sugars and amino acid cores. As will be appreciated, a nucleic acid mimic may comprise base moieties having different polymerization scaffolds (e.g., some scaffolds may comprise a sugar, others may comprise an amino acid core, etc.). Preferred linkage chemistries include phosphothoiate linkages, a phosphodiester linkages, phosphonate linkages, and peptide bonds.

After isolating a structuremer/target complex, it is preferred to determine the identity of the structuremer in the complex. Typically, this is accomplished by separating the structuremer from the non-nucleic acid target molecule in the structuremer/target complex. The structuremer can then be identified. In preferred embodiments, the structuremer is identified by combining the structuremer with a plurality of different species of single-stranded nucleic acid molecules under conditions that allow hybridization between a structuremer and a nucleic acid molecule substantially complementary thereto to form a structuremer/nucleic acid complex. In other embodiments, the identity of the structuremer is determined by deducing the linear sequence of base moieties (or other moieties) used to synthesize the structuremer.

In embodiments where the identity of the structuremer is determined by hybridization to a nucleic acid molecule that is substantially complementary to the bases of the nucleic acid mimic, the nucleotide sequence of the nucleic acid is determined. If desired, the substantially complementary nucleic acid molecule can first be amplified. In addition, or in the alternative, it may be cloned into a suitable vector, which is then used to transform a host cell. Thereafter, nucleic acids can be isolated from the host cell and the nucleotide sequence of the nucleic acid molecule substantially complementary to the structuremer is determined.

Another aspect of the invention relates to structuremers, including those identified according to the methods of the invention.

As those in the art will appreciate, the following detailed description describes certain preferred embodiments of the invention in detail, and is thus only representative and does not depict the actual scope of the invention.

### DETAILED DESCRIPITON OF THE INVENTION

Before the present invention is described in detail, it is understood that the invention is not limited to the particular structuremers and methodology described, as these may vary. It is also to be understood that the terminology used herein is for the purpose of describing particular embodiments only, and is not intended to limit the scope of the invention defined by the appended claims.

1. Introduction.

The present invention concerns methods for isolating and identifying compounds that specifically bind to target molecules other than naturally nucleic acids, including biologically relevant molecules such as proteins, carbohydrates, lipids, and metabolites. The methods employ a class of compounds referred to as “structuremers.”

2. Structuremers.

Structuremers are molecules that specifically associate with non-nucleic acid target molecules. Structuremers preferably comprise a nucleic acid mimic capable of hybridizing to a substantially complementary single-stranded nucleic acid molecule under stringent hybridization conditions. In preferred embodiments, nucleic acid mimics are polymers assembled from on eor more base moiety species (each of which preferably comprises a polymerization scaffold linked to a base (e.g., adenine, guanine, cytosine, thymine, uracil, inosine, xanthine, and hypoxanthine, or a heterocyclic derivative, analogue, or tautomer thereof) capable of specific hybridization to one or more complementary bases in a single-stranded nucleic acid molecule. The nucleic acid mimics tend to be non-amplifiable because the polymerization scaffolds of at least some of the monomeric building blocks used to assemble the structuremer are linked via non-natural linkage chemistries (i.e., other than phosphodiester bonds), and/or because the polymer includes one or more non-naturally occurring bases. Structuremers may further comprise other elements, such as targeting elements comprised of L- and/or D- amino acids linked by peptide or other linkages, as well as one or more other nucleic acid mimics (of the same or different species). One or more tagging moieties are also preferably included in the structuremers, for example, to facilitate separation. The secondary and tertiary structures of the structuremers, as well as their electrostatic charge, hydrophobicity, etc., dictate which, if any, target molecules they will specifically associate with. As will be appreciated, once a structuremer is identified that is specific for a target molecule, its specificity and/or selectivity can be optimized or otherwise refined by one or rounds of optimization.

Structuremers, and their component parts, particularly nucleic acid mimics and, if included, targeting elements, are preferably synthesized using solid-state chemistries. The molecules may be assembled from monomeric units (e.g., individual base moieties), or by pre-assemblying monomeric units into short polymers that can later be linked together to create the desired polymer. In preferred embodiments, the synthesis procedures randomly incorporate one of several different monomeric units (or pre-assemblages of monomeric units) at a given position in the growing polymer chain. In this way, a diverse combinatorial library of different structuremers can readily be synthesized for use in screening against a desired target molecule species in accordance with the instant methods. After a target-specific structuremer is identified, one or iterative rounds of optimization can be undertaken in order to enhance or otherwise alter one or more of the properties of the then-current generation of target-specific structuremer.

A. Nucleic Acid Mimics.

Preferred nucleic acid mimics are polymers of base moieties. Moieties other than base moieties (e.g., polymerization scaffolds that are not linked to a base) may also be included in a nucleic acid mimic, in addition to base moieties. Preferably, in embodiments where one or more other such moieties are included between two base moieties, the spatial relationship of the hydrogen bond donors and acceptors of the bases in the nucleic acid mimic are not disrupted to the extent that they are unable to base pair, i.e., form hydrogen bonds with, with complementary bases in a single-stranded polynucleotide in a duplex.

Nucleic acid mimics may be assembled from monomeric units (ie., individual base moieties), or from multimeric subunits comprising a plurality of base moieties. A “monomeric unit” refers to a single base moiety. A “multimeric subunit” or “pre-assemblage” refers to polymer of several base moieties. They may be readily synthesized using standard solid state of solution chemistry techniques. Preferably, they contain 2 to about 50, most preferably 2 to about 20, base moieties. As will be appreciated, nucleic acid mimics can readily be assembled by linking two or more multimeric subunits using suitable chemistries. For instance, linkage can be directly between a reactive group in each of two polymerization scaffolds, or indirectly, such as by a straight-chain linker attached at one end to a polymer-forming reactive group on one polymerization scaffold and by the other end to a polymer-forming reactive group on the next polymerization scaffold.

The polymerization scaffold of a base moiety can be any suitable chemical that can be linked to a nucleotide-binding base polymerized to the polymerization scaffolds of other base moieties, linker moieties, or other molecules using suitable chemistries, preferably sequential solid state chemistry methods. Preferred polymerization scaffolds are sugars, such as ribose and deoxyribose, and amino acid cores.

As will be appreciated, with respect to linear nucleic acid mimics, a base moiety found at the proximal end will be linked only to a succeeding base moiety, and a base moiety found at the distal end will be linked only to a preceding base moiety. For purposes of the invention, the “proximal end” of a linear nucleic acid mimic will be one end, or terminus, of the molecule, and the “distal end” will mean the other end, or terminus, when viewed along the axis made by the sites of the polymerization scaffolds that are used to link the individual base moieties together. Polymerization scaffolds used to make base moieties may also contain additional sites that may be derivatized. Also, one or more of the sites that can be derivatized may be “protected” (i.e., capped with a chemical group that makes the site non-reactive under certain chemical conditions, e.g., the conditions used for polymerization of base moieties), meaning that in order for it to be derivatized, it must first be “deprotected” (i.e., the protective group must first be removed). Any suitable chemistry now known or later developed for protection and deprotection of reactive sites can be employed.

In a base moiety, the base is bonded to the polymerization scaffold, directly or through a linker moiety. In the broadest sense, a base preferably is a molecule that comprises a set of hydrogen bond donors and acceptors arrayed in a manner that, when juxtaposed to the hydrogen bond donors and acceptors of a nucleotide that participate in hydrogen bond formation with a complementary nucleotide in a nucleic acid duplex, form analogous hydrogen bonds. Preferred examples of bases for inclusion in base moieties include those found in naturally occurring nucleic acids, namely adenine, cytosine, guanine, thymine, and uracil, as well as other bases such as inosine and other purine and pyrimidine analogs. While it is preferred that a base be capable of forming one or more hydrogen bonds that can participate in base pairing in a duplex formed between a structuremer and a substantially complementary polynucleotide, in some cases the base may serve simply as a spacer which does not participate in hydrogen or other forms of bonding with a single-stranded polynucleotide. When a base moiety including such a “spacer” moiety (or a base moiety having no base, only a scaffold) is incorporated into a structuremer, the spacer moiety may participate in interactions with a target molecule. Thus, while a structuremer will contain a sufficient number of base moieties having bases that can participate in the formation and stabilization of a duplex with a substantially complementary nucleic acid, it may also contain one or more spacer moieties interspersed among the other base moieties.

Nucleic acid mimics (and nucleic acids) useful in practicing the present invention can be prepared by any suitable method, now known or later developed. Preferably, such molecules are synthesized by solid-state chemical methods, although in solution chemistries may also be used.

Nucleic acid mimics preferably contain modified chemical groups to enhance their performance or to facilitate characterization. For example, backbone-modified oligonucleotides such as phosphorothioates or methylphosphonates can resist nucleolytic activity, as is the case with PNAs; other modifications include methylphosphonates, monothiophosphates, dithiophosphates, phosphoramidates, phosphate esters, bridged phosphoroamidates, bridged phosphorothioates, bridged methylenephosphonates, dephospho intemucleotide analogs with siloxane bridges, carbonate bridges, carboxymethyl ester bridges, acetamide bridges, carbamate bridges, thioether, sulfoxy, sulfono bridges, and borane derivatives. It has been observed, however, that many of these modified backbones have led to decreased stability for hybrids formed between the modified oligonucleotide and its complementary native oligonucleotide, as assayed by measuring Tm values. Consequently, it is generally understood that backbone modifications destabilize such hybrids, ie., result in lower Tm values, thereby limiting their utility in the context of nucleic acid detection and analysis. As will be appreciated, nucleic acid mimics may also contain mixtures of modified backbones and natural nucleotides. For example, the mimic may comprise a PNA portion and a phosphorothioate portion.

Nucleic acid mimics may additionally comprise substituents other than base moieties to add length and or altered target specificity to the polymer. For example, non-nucleotide monomeric units (see, e.g., U.S. Pat. No. 6,031,091) built around a linker having two coupling groups for adding individual units (or sub-assemblies comprising several already-polymerized units) serially to a growing polymer by a suitable chemistry, or combination of chemistries. Such linkers typically further comprise another covalently attached group, e.g., a base, a label, etc. Such non-nucleotide monomeric units do not appreciably contribute to the ability of a nucleic acid mimic to hybridize to a substantially complementary probe oligonucleotide under stringent conditions.

The binding properties of structuremers to target molecules are dependent in part on their secondary structures. Chemical modifications to the one or more structuremer species in synthesized structuremer libraries can be used to increase the variety of secondary structures. Such modifications include linkers, spacers, and amino acids, all of which may be used to introduce specific funnctionalities to nucleic acid mimics. Such functionalities may also contribute to a better solubility or various degree of cross-linking of nucleic acid mimics.

i. PNAs.

Peptide nucleic acids represent a particularly preferred class, or genus, of nucleic acid mimics for inclusion in structuremers. Broadly, PNAs are amino acid-based compounds comprising ligands other than R-groups found in naturally occurring amino acids that are linked to a peptide backbone rather than to a sugar-based phosphodiester backbone, as is found in naturally-occurring nucleic acids. A “peptide backbone” refers to a series of base moieties (or base moieties and other units having a polymerization scaffold but not a base) linked via peptide bonds, and thus comprises a series of amino acid cores linked via peptide bonds. PNA-based structuremers are structuremers wherein at least some the R-groups attached to the amino acid cores are bases that can hybridize to complementary bases in a single-stranded nucleic acid. In some preferred embodiments, all of the R-groups comprise such bases. In others, some of the R-groups are bases, while other R-groups are chemical groups other than bases, some, none, or all of which may contribute to hybridization with single-stranded nucleic acids.

Representative PNA ligands may include one or more of any of the five main naturally-occurring bases (i.e., thymine, uracil, cytosine, adenine, or guanine), other naturally occurring bases (e.g., 1 -methylguanine, N2-dimethylguanine, N6-dimethyladenine, inosine, uracil, 5-methylcytosine, 5-hydroxymethylcytosine, or thiouracil), and/or non-naturally-occurring bases e.g., bromothymine, azaadenines, azaguanines, etc.) attached to a peptide backbone through a suitable linker. PNAs are able to bind complementary single-stranded nucleic acids. PNAs can be synthesized by any suitable method, or combination of methods. Synthetic methods can be performed in solution or by solid-state methods. PNAs can be assembled from individual monomeric units (e.g., base moieties), or by combining sub-assemblies each comprised of two or more polymerized monomeric units. Preferred methods for making and using PNAs are described in U.S. Pat. No. 5,539,082.

In particularly preferred embodiments of PNAs, the backbone of the nucleic acid mimic comprises repeating N-(2-amino-ethyl) glycine units. Nucleotide bases are connected to each repeating amino acid, via a methylene carbonyl linker attached to the glycine amino group. 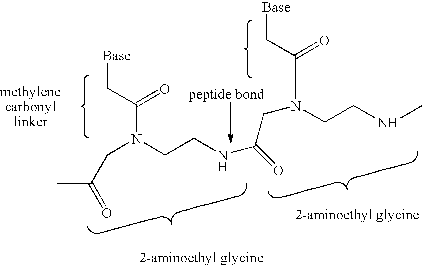
Each PNA monomer participates in two amide bonds, except for those at the proximal and distal ends, which each participate in one such bond. Each monomer (or sub-assembly of several monomers) is sequentially linked via an amide bond formed between a glycine carboxyl group and a 2-amino group of N-(2-amino-ethyl) glycine. When a plurality of such units are connected, the result as an uncharged, achiral DNA or RNA mimic. A structural comparison between DNA and PNA is depicted below:

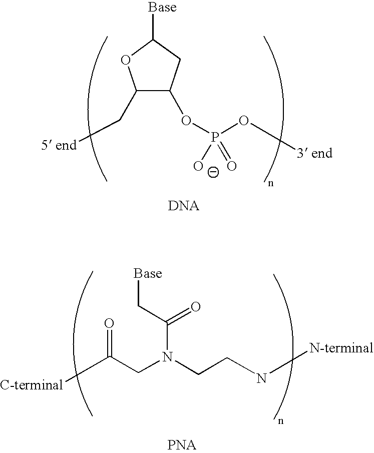

PNAs are chemically stable and resistant to degradation, even inside living cells. In general, because there is no direct interaction between PNA and either DNA polymerase or reverse transcriptase, PNAs are non-amplifiable. In fact, inclusion of a PNA portion in an amplification primer inhibits the elongation of the primer.

PNA-based structuremers can also be designed to form various secondary structures, such as hairpins. Any suitable chemical modification to PNAs can be used, including the use of functionalized backbones and non-natural nucleobases. In particular, but not exclusively, molecules with pendant or enchained amino acids are of interest for this invention. It is also known that two PNA molecules can form a base-paired helical duplex, with the preferred helical sense induced by a terminal chiral amino acid. The propagation of helicity depends on the base pairs closest to the chiral center, and the choice of amino acid is crucial for the sense of helicity. Also, PNAs can be modified by amino acids within the chain or at one or both ends to, for example, increase aqueous solubility, as well as multiply structural variation between a pluralities of different PNA species.

PNAs may be synthesized using combinatorial methods. The length of combinatorially synthesized PNAs preferably ranges from between about 5 to about 50 base moieties, more preferably between about 15 and 40 bases. As preferred synthetic methods involve the serial additions of one base at a time, using a combinatorial approach it is possible to add one of two or more different bases at each position. For example, when a structuremer is being synthesized using base moieties wherein the base is one of the four bases that naturally occur in DNA (i.e., A, G, C, and T), at one, some, or all of the positions of the nucleic acid mimic it is possible to direct that a different base be added to that position in each of four separate reactions. Thus, in this example, nucleic acid mimics can be generated combinatorial means using all four or a limited number (3 or 2) of bases. As will be appreciated, restricting the number of combinatorial events (not all positions and/or less than all possible bases at one position) reduces the complexity of the final reaction solution. This could be advantageous with regard to the sensitivity of the approach. For example, if one of structuremer species in the pool of many structuremer species used in the initial screen is specific for the target molecule, but only has a relatively low affinity for the target molecule, the formation of structuremer/target complexes might be at too low a frequency (i.e., there are too few of such complexes) to detect or be isolated.

As with other nucleic acid mimics, PNAs can be synthesized by combining pre-assemblages of base moieties. Also, a PNA in one structuremer may be chemically linked (e.g., via a linker) to another nucleic acid mimic. As a result, dimer, trimers, and higher order multimers may be assembled from the same or different PNA species. Such linking can be facilitated by including appropriate base moieties having suitable conjugation partners. The addition of affinity tags to structuremers, as well as target molecules, facilitates purification steps of PNA/nucleic acid complexes (second combinatorial step). Such affinity tags include be biotin, glutathione, oligo histidyl, haptens, etc. It is preferred, however that if various structuremer species and the target molecule used in a screening assay for structuremer isolation each includes a tag, it is preferred that the tags be different. In this way, use of the tag on the target molecule to isolate structuremer/target complexes does not also result in the isolation of free structuremers incorporating the same tag.

3. Methods of Isolating and Identifying Structuremers.

In general, to identify a target-specific structuremer, a mixture containing a plurality different structuremer species (e.g., more than 10⁶ species) is combined with the desired non-nucleic acid target molecule under physiological conditions. Structuremer/target complexes, if any, are then separated from other reaction components. To facilitate separation, the structuremer preferably includes a tag. Isolation is performed, for example, in batch mode or using column chromatography. Conditions should preferably be physiological. UV spectroscopy or other methods can be used to monitor the efficiency of washing steps and final completion of purification. A series of wash steps with increasing stringency may also be used.

The resulting structuremer/target complexes are then combined with a library (e.g., more than 10⁶ species) of different single-stranded nucleic acid molecules, or “detection nucleic acid molecules” (e.g., oligonucleotides), under conditions that allow hybridization between the nucleic acid mimic portion of the structuremer with a nucleic acid substantially complementary thereto to form a structuremer/nucleic acid complex. While it is preferred that the structuremer be probed with a library of nucleic acids that is fully representative (i.e., the library contains a sufficient number of species to single-stranded nucleic acids to ensure that at least one species is perfectly complementary to the base sequence (which may include one or more moieties that are not “bases”, but may include hydrogen bond donors and/or acceptors that may participate in hydrogen bond formation with the base of a nucleic acid juxtaposed thereto in a duplex formed between the two molecules, although it need not), libraries of nucleic acids that are less than fully representative may also be employed.

Before hybridization, it is preferred that the structuremers be dissociated from target molecules by any suitable method, for example, by heat or by treatment with acid or alkaline solutions. If acid or base is used to effect dissociation, it is preferred that the pH of the structuremer-containing solution be adjusted before adding the combinatorial library of oligonucleotides. Hybridization is performed under suitable conditions. Preferably, the incubation temperature is a few degrees lower than the average melting temperature of the structuremers in the library used for screening.

The nucleic acid component of the complex is preferably then sequenced (often after separation and amplification steps), thereby allowing the sequence of the nucleic acid mimic portion of the structuremer to be deduced. In those cases when separation and amplification steps are desired, they may be performed using any suitable method. For instance, solid-phase purification of structuremer/nucleic acid complexes can be accomplished using a high affinity binding pair, e.g., a biotin molecule linked to the structuremer and streptavidin linked to a solid support. After incubating the solution containing the structuremer/nucleic acid complexes with the solid support under conditions that allow the members of the binding pair to come together, the solid support is washed several times, with each typically increasing in stringency (e.g., each wash having a lower salt concentration than the previous wash) so as to elute non-specifically bound molecules from the support. After finishing the wash procedure, the structuremer/nucleic acid complexes can be amplified.

In vitro amplification systems that employ enzymatic amplification are preferred. Generally, conventional enzymatic amplification schemes can be broadly grouped into two classes based on whether the amplification reactions are driven by continuous cycling of the temperature between the denaturation temperature, the primer annealing temperature, and the amplicon (i.e., the product of enzymatic amplification of nucleic acid) synthesis temperature, or whether the temperature is kept constant throughout the enzymatic amplification process (isothermal amplification). Typical cycling nucleic acid amplification technologies (thermocycling) are polymerase chain reaction (PCR) and ligase chain reaction (LCR). Specific protocols for such reactions are discussed in, for example, Short Protocols in Molecular Biology, 2.sup.nd Edition, A Compendium of Methods from Current Protocols in Molecular Biology, (Eds. Ausubel et al., John Wiley & Sons, New York, 1992) chapter 15. Reactions which are isothermal include transcription-mediated amplification (TMA), nucleic acid sequence-based amplification (NASBA), and strand displacement amplification (SDA). U.S. Patent documents which discuss nucleic acid amplification include U.S. Pat. Nos. 4,683,195; 4,683,202; 5,130,238; 4,876,187; 5,030,557; 5,399,491; 5,409,818; 5,485,184; 5,409,818; 5,554,517; 5,437,990 and 5,554,516. It is well known that methods such as those described in these patents permit the amplification and detection of nucleic acids without requiring cloning.

Prior to amplification, the structuremer/nucleic acid complexes are dissociated, e.g., using 0.1 M sodium hydroxide or heat. Oligonucleotide primers and the other reagents needed for amplification of the nucleic acids specific for the structuremer(s) are then added, if not already present in the reaction mixture. The oligonucleotides contained in the library of nucleic acids used to probe the structuremers also contain the primer binding sequences that correspond to the amplification primers to be used. Preferably, when two primers are used, they contain different nucleotide sequences. The primers should also include features (e.g., restriction enzyme recognition sites, motifs that facilitates non-template driven addition of adenine at the 3′-ends of PCR strands by the thermostable polymerase (see, e.g., Brownstein, et al. BioTechniques, 20: 1008-1010, 1996, which describes “PIG-tailing,” which facilitates efficient T/A cloning), etc.) that facilitate subsequent cloning steps. Amplification can be performed using any suitable method. One such method is PCR. A representative set of conditions for PCR amplification in a standard thermocycling device are: initial denaturation at 94-99° C. for 10 sec. to 4 min., preferably at 95° C. for 1 min., followed by 20-40 three-step cycles of: 94-99° C. for 10 sec. to 1 min. (preferably at 95° C. for 20 sec.), then at 52-65° C. for 10 sec. to 1 min. (preferably at 55° C. for 20 sec.), and then at 70-74° C. for 10 sec. to 1 min. (preferably at 72° C. for 20 sec.). In preferred embodiments that employ PCR, after amplification, an adenine is added so that the resultant PCR products can be efficiently subcloned into T/A vectors (available, for example, from Promega and Invitrogen) especially suited for PCR products with one 3′-overhanging adenine on each side of the double-stranded amplification product. Ligation of the vector and inserts is performed according to the ligase supplier's directions.

After the ligation reactions are complete, the ligated vectors may be introduced into suitable host cells by any suitable technique (e.g., transformation). Bacterial (e.g., MC1016), or another suitable host microorganism, should be made be competent before transformation. A variety of methods are available, e.g., treatment with CaCl₂ or electroporation. Competent cells can also be purchased from commercial sources and used according to the manufacturer's recommendation. Transformation of CaCl₂-treated cells is typically done for 30 min. on ice and 30 min. at 37° C. After transformation, cells are immediately spread on agar plates (which typically contain a selectable marker, e.g., any of a number of suitable antibiotics, for which the vector encodes a resistance gene) in a dilution suitable to obtain single colonies after overnight incubation at 37° C. Single colonies are picked and grown again overnight in suspension. After preparation of recombinant plasmid DNA using manual procedures or commercially available kit, DNA is denatured and sequenced by conventional methods (e., using a thermocycling sequencing kit (e.g., ABI) and high throughput sequencing instrumentation (e.g., ABI 3700)). Preferably, about 100 clones are sequenced to facilitate subsequent statistical analysis of the sequence data. If, for example, only one structuremer species bound the target molecule, all clones would have the same sequence. However, if non-specific binding occurred, more than one structuremer may have been isolated and identified. Such non-specific events are revealed by the existence of only a few clones having that particular sequence. A representative example of such results for three different screening assays is shown below as Table 1.

TABLE 1

Groups

| | 1 (n) | 2 (n) | 3 (n) | ns |
|---|---|---|---|---|
| Exp 1 | 92 | 0 | 0 | 8 |
| Exp 2 | 38 | 44 | 0 | 18 |
| Exp 3 | 33 | 29 | 35 | 3 |

(n): number of clones having the same sequence;
ns: total non-specific clones

As shown in Table 1, the results for Experiment 1 indicate that only one structuremer specifically bound to the target molecule. Of the 100 clones sequenced, all but eight reveal the same sequence. As the nucleotide sequence of the 92 clones is the same, they correspond to the products amplified different probe oligonucleotides, each of which had the same probe sequence and thus hybridized to the structuremers of the same species. The results of Experiment 2 show that two structuremers specifically bound to the target molecule, whereas in Experiment 3, three structuremers specifically associated with the target. The identity of the structuremer(s) that bound to the target molecules in each of these experiments would comprise a nucleic acid mimic complementary to the probe oligonucleotide used. Based on the knowledge of which bases (or bases) was added at a given position in the structuremer as it was being synthesized, the identity of the target-specific structuremers could be deduced.

In this way, initial lead compounds that specifically bind to or otherwise interact with a particular target molecule can rapidly be identified. If desired, subsequent rounds of derivatization of the initial structuremer may be performed to produce compounds with improved characteristics, for example, improved target specificity and/or selectivity, greater binding affinity, enhanced stability, greater drug-likeness, improved ADME characteristics, etc. Accordingly, these methods are valuable not only in the context of drug discovery, but also to identify reagents useful in diagnostics, protein purification, and other areas where analyte-specific binding is useful.

4. Applications.

Structuremers of the present invention will be useful for research and commercial applications, including diagnostic and therapeutic applications. The variety of different applications include inhibition/activation of enzymes, receptors, bacterial growth, and virus replication. Since non nucleic acid mimics are not metabolized, adverse drug reactions often caused by toxic intermediate metabolites can be avoided. Other applications include in diagnostics, where structuremers may be used in the role now played by antibodies. Structuremers may also be used as affinity reagents, for example, in the purification of target molecules specifically bound by the structuremer.

All patents and patent applications, publications, scientific articles, and other referenced materials mentioned in this specification are indicative of the levels of skill of those skilled in the art to which the invention pertains, and each of which is hereby incorporated by reference to the same extent as if each reference had been incorporated by reference in its entirety individually. Applicants reserve the right to physically incorporate into this specification any and all materials and information from any such patents and patent applications, publications, scientific articles, electronically available information, and other referenced materials or documents.

The specific methods and products described in this specification are representative of preferred embodiments and are exemplary and not intended as limitations on the scope of the invention, and it is understood that the terminology used herein is for the purpose of describing particular embodiments only and is not intended to limit the scope of the present invention which will be limited only by the appended claims. Other objects, aspects, and embodiments will occur to those skilled in the art upon consideration of this specification and are encompassed within the spirit of the invention as defined by the scope of the claims. It is understood that this invention is not limited to the particular materials and methods described, and it will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. Also, the terms “comprising”, “including”, “containing”, etc. are to be read expansively and without limitation. It must be noted that as used herein and in the appended claims, the singular forms “a”, “an”, and “the” include plural reference unless the context clearly dictates otherwise.

The terms and expressions that have been employed are used as terms of description and not of limitation, and there is no intent in the use of such terms and expressions to exclude any now-existing or later-developed equivalent of the features shown and described or portions thereof, but it is recognized that various modifications are possible within the scope of the invention as claimed. Thus, it will be understood that although the present invention has been specifically disclosed by preferred embodiments and optional features, modification and/or variation of the disclosed elements may be resorted to by those skilled in the art, and that such modifications and variations are within the scope of the invention as claimed.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims. In addition, where features or aspects of the invention are described in terms of Markush groups, those skilled in the art will recognize that the invention is also thereby described in terms of any individual member or subgroup of members of the Markush group.

1-16. (canceled)

17. A method for identifying a structuremer specific for a non-nucleic acid target molecule, comprising:
a. isolating a structuremer/target complex formed in a reaction mixture that comprises a structuremer and a non-nucleic acid target molecule under conditions that allow a target-specific structuremer to associate with the non-nucleic acid target molecule;
b. separating the structuremer from the non-nucleic acid target molecule in the structuremer/target complex; and
c. identifying the structuremer,
wherein identification of the structuremer comprises (i) combining the structuremer with a plurality of different species of single-stranded nucleic acid molecules under conditions that allow hybridization between a structuremer and a nucleic acid molecule substantially complementary thereto to form a structuremer/nucleic acid complex; and (ii) determining the nucleotide sequence of a nucleic acid molecule that is substantially complementary to the structuremer.

18. A method according to claim 17 wherein the nucleotide sequence of the nucleic acid molecule that is substantially complementary to the structuremer is determined by a method comprising:
a. separating the structuremer from the nucleic acid molecule in the structuremer/nucleic acid complex; and
b. sequencing the nucleic acid molecule.

19-20. (canceled)
